(19) United States
(12) Patent Application Publication
(10) Pub. No.: US 2014/0107028 A1
(43) Pub. Date: Apr. 17, 2014

(54) METHODS AND COMPOSITIONS FOR PREDICTING RESPONSE TO GLP-1 ANALOGS

(71) Applicant: The University of Chicago, Chicago, IL (US)

(72) Inventors: Sindhu RAJAN, Chicago, IL (US); Louis H. Philipson, Chicago, IL (US); John B. Ancsin, Chicago, IL (US)

(21) Appl. No.: 13/795,320

(22) Filed: Mar. 12, 2013

Related U.S. Application Data
(60) Provisional application No. 61/713,870, filed on Oct. 15, 2012.

(51) Int. Cl. G01N 33/74

(52) U.S. Cl. CPC G01N 33/74
USPC 514/7.2; 435/7.21; 435/6.13; 435/6.12

(57) ABSTRACT

Methods and compositions are provided for evaluating a diabetic patient for treatment with an incretin-based therapy. In certain embodiments, evaluation is ongoing and the patient is evaluated at multiple times. In additional embodiments, a diabetic patient is treated with an incretin-based therapy after the patient has been evaluated for expression levels of GLP-1R in a biological sample. In some aspects, the biological sample is specifically a blood sample that has been enriched for peripheral blood monocytes.

This application claims priority to U.S. Provisional Patent Application No. 61/713,870 filed on Oct. 15, 2012, the entirety of which is incorporated herein by reference.

### BACKGROUND OF THE INVENTION

1. Field of the Invention

The present invention relates generally to the field of medicine. More particularly, it concerns methods and compositions for diabetes care, especially with respect to treatment with incretin-based therapies such as GLP-1 mimics.

2. Description of Related Art

The hallmark of diabetes mellitus, a rapidly growing disease with greater than $200B yearly cost in the United States alone, is elevated blood sugar. Serum glucose concentration is normally maintained within a narrow range principally by the opposing actions of two hormones, glucagon which can raise blood sugar and insulin that can lower blood sugar. While glucagon acts primarily at the liver to mobilize glucose from extensive glycogen stores, insulin acts via its surface receptor to stimulate the cellular uptake of glucose by well-characterized glucose transporters primarily in the liver, muscle and fat. A reduction in insulin receptor signaling can lead to a condition called insulin resistance which can remain undetected for many years since the beta-cells of the pancreas can often compensate for the reduced sensitivity to insulin by increasing insulin synthesis and secretion. Insulin resistance can persist for many years before the loss of glucose control and type-2 diabetes mellitus becomes evident (1). By the time impaired fasting glucose is detected in pre-diabetic patients, as much 40% of their beta-cells may have been lost (2). Glucagon like peptide-1 (GLP-1), an incretin peptide hormone, which is secreted transiently by cells in the small intestine following food intake, has been shown to have a number of anti-diabetic properties including the promotion of beta-cell survival, enhanced insulin secretion, decreased gastric emptying, enhanced satiety with weight loss, and enhanced tissue insulin sensitivity. These activities are dependent on the GLP-1 receptor (GLP-1R), which on binding GLP-1 stimulates a G-protein coupled signal transduction pathway that is susceptible to desensitization. Promising new anti-diabetes treatments have been developed (at least five are currently marketed in the United States) which promote this pro-insulinotropic GLP-1 signaling pathway. These include both direct GLP-1 receptor agonists and indirect enhancers of endogenous GLP-1 activity by inhibiting a key degrading peptidase, DPP4. However, these agents may be ineffectual in some patients at the outset, and in others may lose their effectiveness over time. It is unknown whether drug holidays or intermittent use might improve the clinical effectiveness of these agents. A simple test to evaluate these possibilities and guide therapy is currently lacking.

### SUMMARY OF THE INVENTION

Methods and compositions are provided that are related to evaluating and treating diabetic patients with respect to an incretin-based therapy. An incretin-based therapy involves treatment that involves the GLP-1 pathway. An incretin-based therapy may be one in which treatment is effected with GLP-1 or a GLP-1 analog (compound that mimics GLP-1).

In some embodiments, there are methods of treating a diabetic patient with an incretin-based therapy comprising: a) evaluating a measured expression level of GLP-1R in the patient; and, b) administering the incretin-based therapy using the evaluation to determine the optimum dose and time course of therapy.

In additional embodiments there are methods for evaluating an incretin-based therapy in a patient diagnosed with diabetes or with symptoms of diabetes comprising: a) measuring expression of GLP-1R in a biological sample from the patient to obtain a GLP-1R reference level; b) comparing the GLP-1R reference level to a GLP-1R control level; c) determining the patient has an expression level of GLP-1R that renders the patient likely responsive to an incretin-based therapy; and, d) reporting the GLP-1R reference level of the patient or reporting that the patient has been determined to have an expression level of GLP-1R that renders the patient likely responsive to an incretin-based therapy. The term GLP-1R reference level refers to a level of GLP-1R expression in a patient that may be used to compare against a GLP-1R expression level that is determined from a subsequently obtained biological sample. In certain embodiments, multiple samples are evaluated and measured for GLP-1R expression levels, and one or more of these expression levels is compared to a GLP-1R reference level. In other embodiments a GLP-1R expression level is measured and compared to one or more previously obtained and measured GLP-1R expression levels.

Additional methods are provided for evaluating an incretin-based therapy for a diabetic patient or a patient showing signs of diabetes comprising: a) measuring expression of GLP-1R in peripheral blood monocyte cells from the normal individuals to obtain a GLP-1R control level; b) comparing the GLP-1R reference level of the patient to a GLP-1R control level; c) determining if the patient has an expression level of GLP-1R that renders the patient likely responsive to an incretin-based therapy; and, d) reporting the ratio of GLP-1R patient reference levels to GLP-1R level of the control and/or whether the patient has been determined to have an expression level of GLP-1R that renders the patient likely responsive to an incretin-based therapy.

Other embodiments concern methods for evaluating an incretin-based therapy for a diabetic patient or a patient showing signs of diabetes comprising: a) measuring expression of GLP-1R in peripheral blood monocyte cells from the patient to obtain a GLP-1R reference level; b) comparing the GLP-1R reference level to a GLP-1R control level; c) determining if the patient has an expression level of GLP-1R that renders the patient likely responsive to an incretin-based therapy; and, d) reporting the GLP-1R reference level of the patient and/or whether the patient has been determined to have an expression level of GLP-1R that renders the patient likely responsive to an incretin-based therapy.

In certain embodiments methods include obtaining a biological sample from the patient. In specific embodiments the biological sample is a blood sample. In certain embodiments, the blood sample is obtained using a syringe or butterfly syringe. In particular embodiments, a blood sample is used to measure GLP-1R expression levels. In additional embodiments, peripheral blood monocytes are isolated or purified prior to measuring GLP-1R expression levels.

Incretin-based therapies are used to treat diabetes. Therefore, many embodiments involve patients who have been determined to be diabetic or to be at risk for diabetes (based on family history, medical history, and/or glucose sugar levels). In some cases, the patient has not yet been diagnosed with diabetes. Some embodiments concern a patient who has not been previously evaluated for GLP-1R expression. In other embodiments, the patient has been previously evaluated for GLP-1R expression or a GLP-1R expression has been previously measured. It is contemplated that a patient is evaluated for GLP-1R expression prior to treatment with an incretin-based therapy; treatment may be the initial treatment with the incretin-based therapy or it may be in conjunction with ongoing incretin-based therapy. In certain embodiments, the patient has been previously treated with an incretin-based therapy, or the patient may have been previously treated with a different incretin-based therapy.

Methods and composition concern evaluating, measuring, or assaying GLP-1R expression levels, which refers to directly determining those expression levels. In other embodiments, expression levels may be determined indirectly, such as by obtaining information about the expression levels or reviewing a report; in some cases, a computer is used to obtain that information.

In certain embodiments, expression of GLP-1R is GLP-1R protein expression at the plasma membrane. GLP-1R protein expression may be assayed or measured from peripheral blood monocyte surface expression. In some cases, GLP-1R protein expression is measured using a GLP-1R binding polypeptide, which may be a GLP-1R antibody or a GLP-1R ligand. In specific embodiments, the GLP-1R binding polypeptide is Extendin-4 or a GLP-1R binding fragment thereof. Some methods and compositions involve a GLP-1R binding polypeptide that is labeled. Labels include, but are not limited to, an enzyme, a photon transfer agent, a peptide, a fluorophore, a chromogen, a radioisotope, or a binding polypeptide. It is contemplated that detection may involve multiple agents, and secondary or tertiary agents may be labeled. In other embodiments, the primary agent is labeled and used to measure GLP-1R expression levels.

Some methods involve measuring expression of GLP-1R comprises flow cytometry. In other embodiments, measuring expression of GLP-1R comprises an ELISA assay, a western blot assay, or slot blot assay. In other examples, however, expression of GLP-1R is GLP-1R transcript expression. GLP-1R transcript expression may be measured using hybridization of one or more probes and/or amplification of nucleic acids using one or more primers. In some cases, GLP-1R transcript expression comprising generating a GLP-1R cDNA using a GLP-1R specific primer.

Methods may include evaluating the measured expression level of GLP-1R by comparing the measured expression level to a representative expression level of GLP-1R that is representative of patients responsive to incretin-based therapy. In some embodiments, the measured expression level is reduced compared to the representative expression. In further embodiments, the incretin-based therapy that is administered to the patient is increased in amount relative to the amount the patient would have been administered if the measured expression of GLP-1R was not reduced. Alternatively, in some methods, there is a step of increasing the amount of an incretin-based therapy after the patient is determined to have a reduced expression level of GLP-1R compared to a control level of GLP-1R.

In some embodiments, evaluating the measured expression level of GLP-1R in the patient comprises comparing the expression level of GLP-1R in the patient to a previously measured expression level of GLP-1R in the patient. If the expression level is reduced 5, 10, 15, 20, 25, 30, 35, 40, 45, 50, 55, 60, 65, 70, 75, 80, 85, 90, 95 percent or more, or any range derivable therein, treatment for the patient may be changed or adjusted. A reduction may also be set forth as a-fold reduction of 1, 1.5, 2, 2.5, 3, 3.5, 4, 4. 5, or 5 or more, or any range derivable therein compared to a previously measured level in the same patient or to a base line level measured in the control non-diabetic individuals. In some embodiments, the amount of incretin-based therapy may be increased; alternatively, the treatment may not or no longer be administered. The patient may receive a different therapy in some circumstances.

In certain embodiments, the measured expression level of GLP-1R in the patient is statistically reduced compared to the previously measured expression level of GLP-1R in the same patient. In some embodiments, methods specifically involve multiple measurements being taken from the same patient. The patient may be given incretin-based treatment after 1 or more of the measurements. In some cases, the patient is measured a first time and then given incretin-based treatment, and then the patient is measured at least a second time. In some embodiments, the patient is measured at least 2, 3, 4, 5, 6 or more times. In other embodiments, GLP-1R expression level is measured periodically, such as every 1, 2, 3, 4, 5 weeks and/or 1, 2, 3, 4, 5, 6, 7, 8, 9, 10, 11, 12 months, or any range derivable therein. In certain cases, the patient is evaluated on a bimonthly basis, or 3-4 times a year. The patient may also be measured for GLP-1R expression levels depending on the patient's responsiveness to an incretin-based therapy. If the patient experiences changes in responsiveness, an expression level of GLP-1R may be measured or evaluated.

Methods may involve an incretin-based therapy that is increased in amount relative to a previous amount administered the patient. In some embodiments, the measured expression level of GLP-1R in the patient is not statistically reduced compared to the previously measured expression level of GLP-1R in the patient. In other embodiments, the incretin-based therapy that is administered to the patient is maintained relative to the incretin-based therapy that was administered before the measured expression level was evaluated.

In some embodiments, a GLP-1R expression level is determined, evaluated, measured, or reported using computer software. Methods also include evaluating the biological sample by calculating a score based on the compared expression levels, wherein the score indicates probability that the patient is or is becoming non-responsive to GLP-1R.

In some embodiments, methods include evaluating one or more differential pair values using a scoring algorithm to generate a diagnostic score for incretin based therapy, wherein the patient is identified as having or as not having increased or reduced sensitivity to incretin based therapy based on the score. It is understood by those of skill in the art that the score is a predictive value about the classification of incretin based therapy. In some embodiments, a report is generated and/or provided that identifies the diagnostic score or the values that factor into such a score. In some embodiments, a cut-off score is employed to characterize a sample as likely having increased or reduced sensitivity to incretin based therapy. In some embodiments, the risk score for the patient is compared to a cut-off score to characterize the biological sample from the patient with respect to increased or reduced sensitivity to incretin based therapy.

Methods may involve obtaining from the patient a biological sample, which means the sample is obtained directly from the patient. In other embodiments, a patient's biological sample may be obtained from an entity that is not the patient, such as the doctor, clinician, hospital or laboratory. In certain embodiments, methods involve a blood sample or a lymph fluid sample or solid tissue sample. In some cases, methods involve fixing the tissue sample in formalin and embedding it in paraffin prior to measuring the level of expression of one or more RNAs or diff pair RNAs in the sample. In additional embodiments, the sample is obtained by fine needle aspirate or FNA. In other embodiments, the sample is retrieved from a biopsy, such as a fine needle aspiration biopsy (FNAB) or a needle aspiration biopsy (NAB).

In some embodiments, a patient is determined to have a biological sample indicative of increased or reduced sensitivity to incretin based therapy. The term “indicative of increased or reduced sensitivity to incretin based therapy” means the data indicate that the patient likely has increased or reduced sensitivity to incretin based therapy, where “likely” means “greater than not.” The determination may or may not be based on a diagnostic score that is calculated based on one or more RNA expression levels or diff pair values. In additional embodiments, methods involve determining a treatment for the patient based on one or more diff pair values. In some embodiments, methods include determining a treatment for the patient based on a calculated diagnostic score. In some embodiments, a patient may be suspected of having a increased or reduced sensitivity to incretin based therapy. In other embodiments, the patient may have previously had an elevated blood glucose condition that was then subsequently treated. In still further embodiments, the patient has a familial history of diabetes or pre-diabetic condition. Some embodiments further involve isolating ribonucleic or RNA from a biological sample. Other steps may or may not include amplifying a nucleic acid in a sample and/or hybridizing one or more probes to an amplified or non-amplified nucleic acid. In certain embodiments, a microarray may be used to measure or assay the level of miRNA expression in a sample.

The incretin-based therapy depend on incretin agonists that binds to the incretin receptor. In certain embodiments, the incretin agonist is a GLP-1 mimetic. In specific embodiments, methods concern the GLP-1 mimetic exenatide synthetic (commercially known as Byetta® for the regimen involving a twice daily injection). In particular embodiments, Byetta® is implemented as adjunctive therapy to improve glycemic control in patients with type 2 diabetes mellitus who are taking metformin, a sulfonylurea, a thiazolidinedione, a combination of metformin and a sulfonylurea, or a combination of metformin and a thiazolidinedione, but have not achieved adequate glycemic control. Bydureon is Exenatide for a once weekly injection. Another GLP-1 mimetic is liraglutide (Victoza), which is given via a once-daily injection. In other embodiments, two other mimetics currently in clinical trials, once daily lixisenatide and once weekly albigluride can be used.

In certain embodiments, methods concern a diabetic patient who has Type 2 diabetes. In other embodiments, a diabetic patient has Type 1 diabetes.

Methods also concern treating the patient with an incretin-based therapy. In some embodiments, a patient is treated with an incretin-based therapy after GLP-1R expression is measured in the patient. Treatment may involve administering the incretin-based therapy after GLP-1R expression is measured or assayed in a biological sample from the patient. In certain embodiments, methods further comprise reporting the patient has a reduced level of GLP-1R expression compared to a standardized GLP-1R expression level in non-diabetic individuals. The term “standardized GLP-1R expression level” refers to a level of GLP-1R that is representative of a GLP-1R expression level, such as a normal level. In other embodiments, a standardized level may be a standardized low level of expression compared to a normal level or may be a standardized high level of expression compared to a normal level. Alternatively, in some embodiments, the patient is treated with an incretin-based therapy at an amount higher than the amount the patient would have been administered without measuring GLP-1R expression.

Methods may involve using a probe or primer that is complementary to a cDNA corresponding to all or part of GLP-1R or a probe or primer that is identical to a nucleic acid sequence in a GLP-1R transcript. In certain embodiments, methods also involve generating a GLP-1R cDNA and/or doing quantitative or semi-quantitative PCR to measure the amount of GLP-1R expression levels. Additional methods may involve using probes consisting of biotinylated and fluorescently tagged GLP-1 derivatives to measure the amount of GLP-1R cell surface expression.

In other embodiments, these methods can be used to quantify cell surface expression of other G-protein coupled receptors, including GIP-R (Glucose dependent Insulinotropic Receptor.

Any of the methods described herein may be implemented on tangible computer-readable medium comprising computer-readable code that, when executed by a computer, causes the computer to perform one or more operations. In some embodiments, there is a tangible computer-readable medium comprising computer-readable code that, when executed by a computer, causes the computer to perform operations comprising: a) receiving information corresponding to a level of expression in a blood sample from a patient of at least GLP-1R, and b) determining a value using information corresponding to the expression level, the value being indicative of whether the patient is responsive, is not responsive, or is becoming non-responsive to an incretin-based treatment protocol.

A processor or processors can be used in performance of the operations driven by the example tangible computer-readable media disclosed herein. Alternatively, the processor or processors can perform those operations under hardware control, or under a combination of hardware and software control. For example, the processor may be a processor specifically configured to carry out one or more those operations, such as an application specific integrated circuit (ASIC) or a field programmable gate array (FPGA). The use of a processor or processors allows for the processing of information (e.g., data) that is not possible without the aid of a processor or processors, or at least not at the speed achievable with a processor or processors. Some embodiments of the performance of such operations may be achieved within a certain amount of time, such as an amount of time less than what it would take to perform the operations without the use of a computer system, processor, or processors, including no more than one hour, no more than 30 minutes, no more than 15 minutes, no more than 10 minutes, no more than one minute, no more than one second, and no more than every time interval in seconds between one second and one hour.

Some embodiments of the present tangible computer-readable media may be, for example, a CD-ROM, a DVD-ROM, a flash drive, a hard drive, or any other physical storage device. Some embodiments of the present methods may include recording a tangible computer-readable medium with computer-readable code that, when executed by a computer, causes the computer to perform any of the operations discussed herein, including those associated with the present tangible computer-readable media. Recording the tangible computer-readable medium may include, for example, burning data onto a CD-ROM or a DVD-ROM, or otherwise populating a physical storage device with the data.

Also provided are kits containing the disclosed compositions or compositions used to implement the disclosed methods. In some embodiments, kits can be used to evaluate GLP-1R transcript or cDNA molecules. In certain embodiments, a kit contains, contains at least, or contains at most 1 probe or primer set. In other embodiments, kits or methods may involve 1, 2, 3, 4, 5, 6, 7, 8, 9, 10, 11, or 12 or more (or any range derivable therein) probes or primers or primer sets directed at GLP-1R.

As used herein the specification, “a” or “an” may mean one or more. As used herein in the claim(s), when used in conjunction with the word “comprising”, the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the method being employed to determine the value, or the variation that exists among the study subjects.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIG. 1 (A-C): Glucose dependent cell surface trafficking of incretin receptors in MING insulinoma and primary beta cells.

FIG. 2 (A-B): Dimished endogenous GLP-1R density at the plasma membrane in MIN6 cells exposed to high glucose concentrations detected by a cell surface biotinylation assay followed by western blot with anti-GLP-1R antibody.

FIG. 3: (A-B): Imaging of glucose dependent trafficking of GLP-1R in MIN6 insulinoma cells with fluorescein tagged agonist.

FIG. 4: Imaging of glucose dependent trafficking of GLP-1R inmacrophage cells with fluorescein tagged agonist.

FIG. 5: Diminished endogenous GLP-1R density at the plasma membrane in macrophage cells exposed to high glucose concentrations detected by a cell surface biotinylation assay followed by western blot with anti-GLP-1R antibody.

FIG. 6 (A-C): Glucose dependent trafficking of GLP-1R trafficking in human monocytic THP-1 cells detected by an antibody raised against the extra cellular N-terminus of GLP-1R.

FIG. 7 (A-B): Rat peripheral blood monocytes show glucose dependent cell surface expression of GLP-1 receptor.

### DESCRIPTION OF ILLUSTRATIVE EMBODIMENTS

### I. Diabetes

Type 2 diabetes is generally characterized by the inability of pancreatic beta cells to respond to growing metabolic demand (Kahn and Valdez, 2003). The gut derived insulinotropic incretin hormones, Glucagon-Like Peptide 1 (GLP-1) and Glucose-dependent Insulinotropic Polypeptide (GIP) play a crucial role in glucose homeostasis by promoting beta-cell survival, inhibiting glucagon secretion and stimulating insulin secretion in a glucose-dependent manner (Drucker). GLP-1 is secreted transiently in response to oral glucose and is inactivated rapidly by serum Dipeptidyl peptidase 4 (DPP-4) (Hoist and Deacon, 2005). Stable and long acting agonists of GLP-1 receptor (GLP-1R) and DPP4 inhibitors are evolving as powerful therapeutic agents for treating Type 2 diabetes.

Efficacy of incretin based therapies depend on efficient incretin signaling and circulating serum DPP4 levels (Aso et al.). Several studies report impaired GLP-1 responsiveness in hyperglycemia due to a reduced GLP-1R signaling (Fritsche et al., 2000; Xu et al., 2007). Moreover, infused GIP failed to potentiate insulin secretion in diabetic patients (Nauck et al., 1993). The mechanisms that cause impaired incretin responsiveness in diabetes are not well understood.

### II. GLP-1 Receptor Function

A new generation of type-2 diabetes treatments can successfully preserve or simulate the function of GLP-1, an incretin peptide hormone with a wide-range of beneficial effects involving glucose and lipid metabolism, reduction of gastric emptying and augmentation of satiety (Drucker et al., 1987). The impetus for the development of GLP-1 as a therapeutic agent has centered on its glucose-dependent promotion of insulin secretion and inhibition of glucagon secretion. These effects promote improved glycemia and weight loss (Drucker and Nauck, 2006). Perhaps as much as 60% of the overall postprandial insulin response is mediated by GLP-1 and a reduction in GLP-1 response has been reported early in diabetes and may worsen with the progression of the disease (Nauck, 2011).

GLP-1 functions to counter these pro-diabetic changes by promoting beta-cell function and survival, inhibiting glucagon secretion and stimulating insulin synthesis and secretion in a glucose-dependent manner. GLP-1 analogs and specific dipeptidyl pepidase-4 inhibitors (DPP4i), which act to preserve GLP-1 function, have been approved as anti-diabetes drugs. GLP-1 and its analogs act through the glucagon like peptide-1 receptor (GLP-1R), a G protein coupled receptor found on beta-cells and several other cell types including monocytes/macrophages. The exact role of the GLP-1R in monocytes is unclear but its activation by GLP-1 or its analog exendin-4 can reduce its cellular inflammatory state (Shiraishi et al., 2012).

GLP-1 signaling begins with the hormone binding to its G-protein coupled receptor (GLP-1R) followed by an increase in cAMP that leads to a variety of tissue specific effects, including inhibition of glucagon. Therefore, the success of incretin-based therapies depends on abundance of plasma membrane GLP-1Rs and efficient GLP-1R signaling. Several studies report diminished GLP-1 responsiveness in hyperglycemia (Fritsche et al., 2000; Xu et al., 2007) Our recent study showed that impaired forward trafficking of GLP-1R to the plasma membrane induced by hyperglycemia contributes to this process (Rajan et al., 2012). In addition, our results demonstrate that prolonged stimulation of the GLP-1R by the GLP-1 analog, Exendin-4 also resulted in diminished cell surface receptor density. This observation is particularly significant since long-acting agonists of GLP-1R are now available (Victoza, Byetta) and are already being used in type 1 diabetes (Deiss et al., 2011; Varanasi et al., 2011).

Glucose dependent cell surface expression of the GLP-1R is a potential biomarker that can be used to guide and test incretin receptor function and efficacy of incretin based treatments. Many cell types including peripheral blood cells express GLP-1Rs. Our pilot studies show that similar to pancreatic beta cells, both hyperglycemia and chronic exposure to (Xu et al., 2007) GLP-1R agonists cause reduced receptor number at the cell membrane of peripheral blood monocytes.

Methods and compositions concern glucagon-like peptide-1 (GLP-1) mimetics (or receptor agonists), which stimulate glucose dependent insulin secretion from beta-cells only when the glucose level is above the normal range; they cease to act between meals when glucose levels are lowered, thereby eliminating the risk of hypoglycemia. However, GLP-1 agonists cannot be orally administered and require a subcutaneous injection. There is presently only one drug in this class, exenatide synthetic, which is typically administered at a dose regimen of 5 mcg per subcutaneous dose in a 1.2 ml prefilled pen (60 doses) and 10 mcg per subcutaneous dose in a 2.4 ml prefilled pen (60 doses).

Other GLP-1 mimetics include PF-04603629, an exendin-transferrin fusion protein that is a long-acting glucagon-like peptide-1 (GLP-1) mimetic. In other embodiments, there is LY2189265 (LY), which may be administered regularly or in escalating subcutaneous doses ranging from 0.1 to 12 mg. Another mimetic is liraglutide (Victoza), which is given via a once-daily injection. At least two other mimetics, once daily lixisenatide and once weekly albigluride are currently in clinical trials. Methods may concern any of these incretin-based therapies.

Dosages may be reduced or increased in increments of the following after having GLP-1R measured: about, at least about, or at most about 0.01, 0.02, 0.03, 0.04, 0.05, 0.06, 0.07, 0.08, 0.09, 0.1, 0.2, 0.3, 0.4, 0.5, 0.6, 0.7, 0.8, 0.9, 1.0, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, 3.0, 3.1, 3.2, 3.3, 3.4, 3.5, 3.6, 3.7. 3.8, 3.9, 4.0, 4.1, 4.2, 4.3, 4.4, 4.5, 4.6, 4.7, 4.8, 4.9, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, 8.5, 8.6, 8.7, 8.8, 8.9, 9.0, 9.1, 9.2, 9.3, 9.4, 9.5, 9.6, 9.7, 9.8, 9.9, 10.0, 10.5, 11.0, 11.5, 12.0, 12.5, 13.0, 13.5, 14.0, 14.5, 15.0, 15.5, 16.0, 16.5, 17.0, 17.5, 18.0, 18.5, 19.0. 19.5, 20.0, 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 105, 110, 115, 120, 125, 130, 135, 140, 145, 150, 155, 160, 165, 170, 175, 180, 185, 190, 195, 200, 205, 210, 215, 220, 225, 230, 235, 240, 245, 250, 255, 260, 265, 270, 275, 280, 285, 290, 295, 300, 305, 310, 315, 320, 325, 330, 335, 340, 345, 350, 355, 360, 365, 370, 375, 380, 385, 390, 395, 400, 410, 420, 425, 430, 440, 441, 450, 460, 470, 475, 480, 490, 500, 510, 520, 525, 530, 540, 550, 560, 570, 575, 580, 590, 600, 610, 620, 625, 630, 640, 650, 660, 670, 675, 680, 690, 700, 710, 720, 725, 730, 740, 750, 760, 770, 775, 780, 790, 800, 810, 820, 825, 830, 840, 850, 860, 870, 875, 880, 890, 900, 910, 920, 925, 930, 940, 950, 960, 970, 975, 980, 990, 1000, 1100, 1200, 1300, 1400, 1500, 1600, 1700, 1800, 1900, 2000, 2100, 2200, 2300, 2400, 2500, 2600, 2700, 2800, 2900, 3000, 3100, 3200, 3300, 3400, 3500, 3600, 3700, 3800, 3900, 4000, 4100, 4200, 4300, 4400, 4500, 4600, 4700, 4800, 4900, 5000, 6000, 7000, 8000, 9000, 10000 milligrams (mg) or micrograms (mcg) or μg/kg or micrograms/kg/minute or mg/kg/min or micrograms/kg/hour or mg/kg/hour, or any range derivable therein.

### III. Techniques

As discussed below expression of GLP-1R transcripts or proteins may also be tested by molecular biological methods, like two-, three- or four-hybrid-assays, RNA protection assays, Northern blots, Western blots, micro-, macro- and Protein- or antibody arrays, dot blot assays, in situ hybridization and immunohistochemistry, quantitative PCR, coprecipitation, far western blotting, phage based expression cloning, surface plasmon resonance measurements, yeast one hybrid screening, DNAse I footprint analysis, mobility shift DNA-binding assays, gel filtration chromatography, affinity chromatography, immunoprecipitation, one- or two dimensional gel electrophoresis, aptamer technologies, as well as high throughput synthesis and screening methods. Methods for measuring expression of mRNA or transcripts can be accomplished using assays involving hybridization and/or amplification. Hybridization may involve one or more probes or sets of primers. Moreover, assays may involve generating a complementary DNA (cDNA) to a GLP-1R transcript. In certain embodiments, sequencing may be involved, which may or may not be quantitative.

A. Flow Cytometry

In certain aspects, GLP-1R expression can be measured by immunofluorescence flow cytometry of peripheral blood cell samples. In certain embodiments GLP-1R specific antibodies may be used to detect cell surface expression of GLP-1R. GLP-1R antibodies can be directly conjugated to a fluorophore and exposed to purified peripheral blood samples to detect expression of GLP-1R. Single channel or multi-channel flow cytometers capable of detecting the fluorophore of the mentioned GLP-1R antibody can be used.

B. Binding Assays

In certain embodiments, a binding assay can be used to detect and measure GLP-1R expression levels. In such embodiments, GLP-1R binding peptide can be used to directly or indirectly measure GLP-1R expression levels. For example, GLP-1R binding peptides can de directly conjugated to a flourophore and its presence detected by flow cytometry, plate readers or fluorimeters capable of detecting the fluorophore in use. Alternatively, indirect methods of detection may be used, in which a secondary antibody conjugated to a fluorophore is used as a readout of GLP-1R binding peptide recognition.

Certain examples of antibody conjugates are those conjugates in which the antibody is linked to a detectable label. “Detectable labels” are compounds and/or elements that can be detected due to their specific functional properties, and/or chemical characteristics, the use of which allows the antibody to which they are attached to be detected, and/or further quantified if desired.

Among the fluorescent labels contemplated for use as conjugates include Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein Isothiocyanate, HEX, 6-JOE, Oregon Green 488, Oregon Green 500, Oregon Green 514, Pacific Blue, REG, Rhodamine Green, Rhodamine Red, Renographin, ROX, TAMRA, TET, Tetramethylrhodamine, and/or Texas Red, among others.

Antibody conjugates include those intended primarily for use in vitro, where the antibody is linked to a secondary binding ligand and/or to an enzyme (an enzyme tag) that will generate a colored product upon contact with a chromogenic substrate. Examples of suitable enzymes include, but are not limited to, urease, alkaline phosphatase, (horseradish) hydrogen peroxidase or glucose oxidase. Preferred secondary binding ligands are biotin and/or avidin and streptavidin compounds. The use of such labels is well known to those of skill in the art and are described, for example, in U.S. Pat. Nos. 3,817,837; 3,850,752; 3,939,350; 3,996,345; 4,277,437; 4,275,149 and 4,366,241; each incorporated herein by reference.

Yet another known method of site-specific attachment of molecules to antibodies comprises the reaction of antibodies with hapten-based affinity labels. Essentially, hapten-based affinity labels react with amino acids in the antigen binding site, thereby destroying this site and blocking specific antigen reaction. However, this may not be advantageous since it results in loss of antigen binding by the antibody conjugate.

Several methods are known in the art for the attachment or conjugation of an antibody to its conjugate moiety. Some attachment methods involve the use of a metal chelate complex employing, for example, an organic chelating agent such a diethylenetriaminepentaacetic acid anhydride (DTPA); ethylenetriaminetetraacetic acid; N-chloro-p-toluenesulfonamide; and/or tetrachloro-3-6-diphenylglycouril-3 attached to the antibody (U.S. Pat. Nos. 4,472,509 and 4,938,948, each incorporated herein by reference). Monoclonal antibodies may also be reacted with an enzyme in the presence of a coupling agent such as glutaraldehyde or periodate. Conjugates with fluorescein markers are prepared in the presence of these coupling agents or by reaction with an isothiocyanate. In U.S. Pat. No. 4,938,948, imaging of breast tumors is achieved using monoclonal antibodies and the detectable imaging moieties are bound to the antibody using linkers such as methyl-p-hydroxybenzimidate or N-succinimidyl-3-(4-hydroxyphenyl)propionate.

C. Immunoassays

Isolated cells can be lysed and GLP-1R protein levels can be determined by Western blotting methods with GLP-1R specific antibodies. Antibodies of the present invention can be used in characterizing the GLP-1R content of tissues or cells in bodily fluids such as blood, cerebrospinal fluid, urine, prostate fluid or semen through techniques such as RIAs, ELISAs and Western blotting.

Immunoassays can be classified according to the assay type, assay method and endpoint labeling method. These three major criteria for classification that have the greatest influence on the performance of test are, i) the use of a limited (type II) or excessive reagent format (type I), ii) the use of a homogeneous and heterogeneous format, iii) the use of a label or unlabelled assay format and the choice of label. It is contemplated that all these kinds of immunoassays may be employed.

In Type I assay format, where antigen binds to an excess of antibody, the most common method is sandwich assay. In this approach, the first antibody (capture Ab) in excess is coupled to a solid phase. The bound antigen is then detected with a second antibody (indicator Ab) labeled with various indicators such as enzymes, fluorophores, radioisotopes, particles, etc. In this assay, the amount of indicator antibody captured on the solid phase is directory proportional to the amount of antigen in the sample.

In some embodiments, an ELISA assay is particularly contemplated. For example, antibodies to GLP-1R may be immobilized onto a selected surface, for example, a surface such as a microtiter well, a membrane, a filter, a bead or a dipstick. After washing to remove incompletely adsorbed material, it is desirable to bind or coat the surface with a non-specific agent that is known to be antigenically neutral with regard to the test sample, e.g., bovine serum albumin (BSA), casein or solutions of powdered milk. This allows for blocking of non-specific adsorption sites on the immobilizing surface and thus reduces the background caused by non-specific binding of antibody to antigen on the surface.

After binding of antibody to the surface and coating, the surface is exposed to blood, blood plasma, blood serum, urine, prostate fluid or semen. Following formation of specific immunocomplexes between antigens in the blood and the antibody, and subsequent washing, the occurrence and even amount of immunocomplex formation may be determined by subjecting the same to a second antibody having specificity for the antigen. Appropriate conditions preferably include diluting the sample with diluents such as BSA, bovine gamma globulin (BGG) and phosphate buffered saline (PBS)/Tween. These added agents also tend to assist in the reduction of non-specific background. The detecting antibody is then allowed to incubate for from about 2 to about 4 hr, at temperatures preferably on the order of about 25° to about 27° C. Following incubation, the surface is washed so as to remove non-immunocomplexed material. A preferred washing procedure includes washing with a solution such as PBS/Tween or borate buffer.

To provide a detecting means, the second antibody may have an associated label, e.g., an enzyme that will generate a color development upon incubating with an appropriate chromogenic substrate. Thus, for example, one will desire to contact and incubate the second antibody for a period of time and under conditions which favor the development of immunocomplex formation (e.g., incubation for 2 hr at room temperature in a PBS-containing solution such as PBS/Tween®.).

After incubation with the second antibody, and subsequent to washing to remove unbound material, the amount of label may be quantified (e.g., by incubation with a chromogenic substrate such as urea and bromocresol purple or 2,2′-azino-di-(3-ethyl-benzthiazoline)-6-sulfonic acid (ABTS) and hydrogen peroxide in the case of peroxidase as the enzyme label). Quantitation is then achieved by measuring the label, e.g., degree of color generation, e.g., using a visible spectrum spectrophotometer.

Other potential labels include radiolabels, fluorescent labels, dyes and chemiluminescent molecules (e.g., luciferase).

In Type II assay formats, a limited amount of antibody is used (insufficient to bind the entire antigen) a prefixed amount of labeled antigen competes with the unlabeled antigen in test sample for a limited number of antibody binding sites. The concentration of unlabeled antigen in specimen can be determined from the portion of labeled antigen that is bound to the antibody. Since most analyte molecules are not enough large to provide two different epitopes in this method, the response will be inversely proportional to the concentration of antigen in the unknown.

The use of either competitive or immunometric assays requires differentiation of bound from free label. This can be archived either by separating bound from free label using a means of removing antibody (heterogeneous) or modulation of signal of the label when antigen is bound to antibody compared to when it is free (homogeneous).

Most solid phase immunoassays belong to the Heterogeneous Assay category. There are many ways of separating bound from free label such as precipitation of antibody, chromatographic method, and solid phase coupling antibody. Homogeneous assays do not require any of separation step to distinguish antigen bound antibody from free antibody. It has an advantage in automation, and typically is faster, easier to perform, and more cost-effective, but its specificity and sensitivity are lower.

Contacting the chosen biological sample with the first antibody under effective conditions and for a period of time sufficient to allow the formation of immune complexes (primary immune complexes) is generally a matter of simply adding the antibody composition to the sample and incubating the mixture for a period of time long enough for the antibodies to form immune complexes with, i.e., to bind to, any anti-GLP-1R complex. After this time, the sample-antibody composition, such as a tissue section, ELISA plate, dot blot or western blot, will generally be washed to remove any non-specifically bound antibody species, allowing only those antibodies specifically bound within the primary immune complexes to be detected.

In general, the detection of immunocomplex formation is well known in the art and may be achieved through the application of numerous approaches. These methods are generally based upon the detection of a label or marker, such as any of those radioactive, fluorescent, biological and enzymatic tags. U.S. patents concerning the use of such labels include U.S. Pat. Nos. 3,817,837; 3,850,752; 3,939,350; 3,996,345; 4,277,437; 4,275,149 and 4,366,241, each incorporated herein by reference. Of course, one may find additional advantages through the use of a secondary binding ligand such as a second antibody and/or a biotin/avidin ligand binding arrangement, as is known in the art. All prior assays to detect immunocomplexes are based on autologous complexes generated by the patient's own antibodies and antigen. The present invention is different in that the assays of the present invention detect immunocomplexes as a result of a therapeutic approach.

The antigen, antibody or antigen:antibody complex employed in the detection may itself be linked to a detectable label, wherein one would then simply detect this label, thereby allowing the amount of the primary immune complexes in the composition to be determined. Alternatively, the first antibody that becomes bound within the primary immune complexes may be detected by means of a second binding ligand that has binding affinity for the antibody. In these cases, the second binding ligand may be linked to a detectable label. The second binding ligand is itself often an antibody, which may thus be termed a “secondary” antibody. The primary immune complexes are contacted with the labeled, secondary binding ligand, or antibody, under effective conditions and for a period of time sufficient to allow the formation of secondary immune complexes. The secondary immune complexes are then generally washed to remove any non-specifically bound labeled secondary antibodies or ligands, and the remaining label in the secondary immune complexes is then detected.

Further methods include the detection of primary immune complexes by a two step approach. A second binding ligand, such as an antibody, that has binding affinity for the antibody is used to form secondary immune complexes, as described above. After washing, the secondary immune complexes are contacted with a third binding ligand or antibody that has binding affinity for the second antibody, again under effective conditions and for a period of time sufficient to allow the formation of immune complexes (tertiary immune complexes). The third ligand or antibody is linked to a detectable label, allowing detection of the tertiary immune complexes thus formed. This system may provide for signal amplification if this is desired.

As detailed above, immunoassays, in their most simple and/or direct sense, are binding assays. Certain preferred immunoassays are the various types of enzyme linked immunosorbent assays (ELISAs) and/or radioimmunoassays (RIA) known in the art. Immunohistochemical detection using tissue sections is also particularly useful. However, it will be readily appreciated that detection is not limited to such techniques, and/or western blotting, dot blotting, FACS analyses, and/or the like may also be used.

Immunoassays encompassed by the present invention include, but are not limited to, those described in U.S. Pat. No. 4,367,110 (double monoclonal antibody sandwich assay) and U.S. Pat. No. 4,452,901 (western blot). Other assays include immunoprecipitation of labeled ligands and immunocytochemistry, both in vitro and in vivo.

Immunoassays generally are binding assays. Certain useful immunoassays are the various types of enzyme linked immunosorbent assays (ELISAs) and radioimmunoassays (RIA) known in the art. Immunohistochemical detection using tissue sections is also particularly useful.

In one exemplary ELISA, the antibodies are immobilized on a selected surface, such as a well in a polystyrene microtiter plate, dipstick, or column support. Then, a test composition suspected of containing the desired antigen, such as a clinical sample, is added to the wells. After binding and washing to remove non-specifically bound immune complexes, the bound antigen may be detected. Detection is generally achieved by the addition of another antibody, specific for the desired antigen, that is linked to a detectable label. This type of ELISA is known as a “sandwich ELISA”. Detection also may be achieved by the addition of a second antibody specific for the desired antigen, followed by the addition of a third antibody that has binding affinity for the second antibody, with the third antibody being linked to a detectable label.

Variations on ELISA techniques are known to those of skill in the art. In one such variation, the samples suspected of containing the desired antigen are immobilized onto the well surface and then contacted with the antibodies of the invention. After binding and appropriate washing, the bound immune complexes are detected. Where the initial antigen specific antibodies are linked to a detectable label, the immune complexes may be detected directly. Again, the immune complexes may be detected using a second antibody that has binding affinity for the first antigen specific antibody, with the second antibody being linked to a detectable label.

Competition ELISAs are also possible in which test samples compete for binding with known amounts of labeled antigens or antibodies. The amount of reactive species in the unknown sample is determined by mixing the sample with the known labeled species before or during incubation with coated wells. The presence of reactive species in the sample acts to reduce the amount of labeled species available for binding to the well and thus reduces the ultimate signal.

Irrespective of the format employed, ELISAs have certain features in common, such as coating, incubating or binding, washing to remove non-specifically bound species, and detecting the bound immune complexes. These are described as below.

Antigen or antibodies may also be linked to a solid support, such as in the form of plate, beads, dipstick, membrane, or column matrix, and the sample to be analyzed is applied to the immobilized antigen or antibody. In coating a plate with either antigen or antibody, one will generally incubate the wells of the plate with a solution of the antigen or antibody, either overnight or for a specified period. The wells of the plate will then be washed to remove incompletely-adsorbed material. Any remaining available surfaces of the wells are then “coated” with a nonspecific protein that is antigenically neutral with regard to the test antisera. These include bovine serum albumin (BSA), casein, and solutions of milk powder. The coating allows for blocking of nonspecific adsorption sites on the immobilizing surface and thus reduces the background caused by nonspecific binding of antisera onto the surface.

In ELISAs, it is more customary to use a secondary or tertiary detection means rather than a direct procedure. Thus, after binding of the antigen or antibody to the well, coating with a non-reactive material to reduce background, and washing to remove unbound material, the immobilizing surface is contacted with the clinical or biological sample to be tested under conditions effective to allow immune complex (antigen/antibody) formation. Detection of the immune complex then requires a labeled secondary binding ligand or antibody, or a secondary binding ligand or antibody in conjunction with a labeled tertiary antibody or third binding ligand.

“Under conditions effective to allow immune complex (antigen/antibody) formation” means that the conditions typically include diluting the antigens and antibodies with solutions such as BSA, bovine gamma globulin (BGG) and phosphate buffered saline (PBS)/TWEEN (polysorbate). These added agents also tend to assist in the reduction of nonspecific background.

The suitable conditions also mean that the incubation is at a temperature and for a period of time sufficient to allow effective binding. Incubation steps are typically from about 1 to 2 to 4 hours, at temperatures that may be on the order of 25° C. to 27° C., or may be overnight at about 4° C. or so.

After all incubation steps in an ELISA are followed, the contacted surface is washed so as to remove non-complexed material. Washing often includes washing with a solution of PBS/TWEEN,® or borate buffer. Following the formation of specific immune complexes between the test sample and the originally bound material, and subsequent washing, the occurrence of even minute amounts of immune complexes may be determined.

To provide a detecting means, the second or third antibody will have an associated label to allow detection. In some embodiments, this will be an enzyme that will generate color development upon incubating with an appropriate chromogenic substrate. Thus, for example, one will desire to contact and incubate the first or second immune complex with a urease, glucose oxidase, alkaline phosphatase, or hydrogen peroxidase-conjugated antibody for a period of time and under conditions that favor the development of further immune complex formation, e.g., incubation for 2 hours at room temperature in a PBS-containing solution such as PBS-TWEEN™.

After incubation with the labeled antibody, and subsequent to washing to remove unbound material, the amount of label is quantified, e.g., by incubation with a chromogenic substrate such as urea and bromocresol purple or 2,2′-azino-di-(3-ethyl-benzthiazoline-6-sulfonic acid [ABTS] and H₂O₂, in the case of peroxidase as the enzyme label. Quantification is then achieved by measuring the degree of color generation, e.g., using a visible spectra spectrophotometer.

Alternatively, the label may be a chemiluminescent one. The use of such labels is described in U.S. Pat. Nos. 5,310,687, 5,238,808 and 5,221,605

It also is contemplated that the above reagents maybe packaged in a kit that may be produced commercially to measure the soluble antigens, antibodies or antibody:antigen complexes described herein.

D. Amplification

1. Quantitative and Semiquantive PCR

These amplification methods may be employed to evaluate, assay or measure GLP-1R expression level. Semiquantitave PCR is used as described by (Murphy et al., 1993; Salvi et al., 1995). These amplification methods may involve primers or primer pairs that are complementary to the sequence to be detected.

E. Functional Assays

Our studies show that hyperglycemia and/or prolonged stimulation of GLP-1R that leads to elevated intracellular cAMP and increased activity of cAMP dependent PKA results in reduced receptor density in the cell membrane under in vitro conditions. Glucose induced down-regulation of GLP-1R is also observed in peripheral blood cells.

Receptor function can be studied by quantifying cAMP generation followed by agonist stimulation using a cAMP specific ELISA.

In some embodiments, standard cAMP assays may be used to determine GLP-1R stimulation of cAMP. For example, standard scintillation proximity assays (SPA) using SPA scintillation beads or SPA imaging beads from Amersham and radiolabeled cAMP may be used; a signal may be read in a scintillation counter or CCD imager.

Other methods to measure cAMP generated by GLP-1R stimulation include enzyme fragment complementation (EFC). In this method, cellular cAMP competes with cAMP that has been labeled with a small peptide fragment of β-galactosidase for binding to an anti-cAMP antibody. In the presence of free cAMP, antibody sites are occupied, leaving the labeled cAMP free to complement with the enzyme fragment, producing active β-galactosidase for substrate hydrolysis.

cAMP levels may also be determined using Perkin Elmer AlphaScreen technology. The described methodology detects a chemiluminescent signal between donor and acceptor beads. Cellular cAMP competes with a biotinylated cAMP probe recognized by a streptavidin donor and anti-cAMP conjugated acceptor beads.

Conventional fluorescence polarization may also be used to determine cAMP levels in GLP-1R stimulated cells. In this methodology a fluorescently-labeled cAMP is exposed to polarized light. The emission becomes depolarized due to molecular rotation in the period between excitation and emission. When the labeled cAMP is bound to an antibody, the observed depolarization is reduced relative to when it is free in solution.

In other methods homogeneous time-resolved fluorescence can be used to measure cAMP levels. This type of fluorescence measurement is a modification of the fluorescence resonance energy transfer (FRET) method which uses novel donor-acceptor pairs for the labeled cAMP antibody and cAMP molecule designed to overcome the problem of autofluorescence resulting from unbound fluorophores. In this method, the native, cellular cAMP binds to anti-cAMP antibodies labeled with europium cryptate (donor) in the presence of competing cAMP labeled with a modified allophyocyanin dye d2 (the acceptor). The use of cryptate rather than a chelate as its donor molecule raises the activation energy of the transition state, making the complex highly resistant to varying buffer and pH, thus increasing the stability of the signal. The HTRF assay from Cisbio is sensitive and highly reproducible for measuring cAMP production in response to Gs and Gi-coupled GPCR stimulation.

In some embodiments cAMP may be measured by a cAMP-Glo™ Assay, a homogeneous and bioluminescent assay to measure cAMP levels in cells. The cAMP-Glo™ Assay monitors cAMP production in cells in response to the effects of an agonist such as GLP-1R agonist or mimetic on G protein-coupled receptors (GPCR). The assay is based on the principle that cyclic AMP (cAMP) stimulates protein kinase A (PKA) holoenzyme activity, decreasing available ATP and leading to decreased light production in a coupled luciferase reaction.

### IV. Examples

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Incretin Receptor Trafficking to the Cell Membrane of MIN6 Insulinoma and Primary Beta Cells is Glucose Dependent

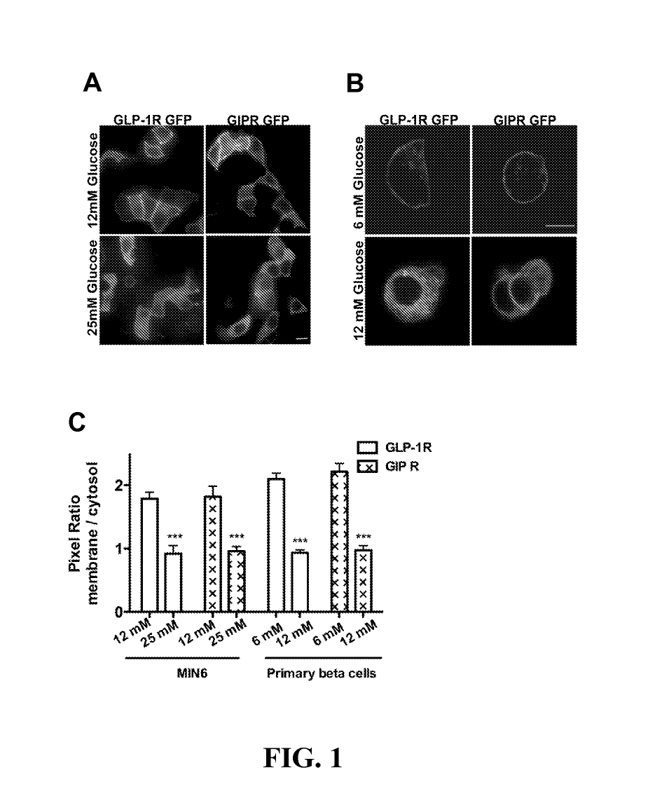

MIN6 insulinoma cells and primary beta cells were transfected with C-terminally GFP tagged GLP-1R or GIPR. Primary beta cells were co-transfected with mouse insulin promoter-GFP to identify beta cells. Under high glucose conditions GFP fluorescence was found to be predominantly intracellular. MIN6 cells transfected with GLP-1R-GFP or GIPR-GFP and imaged 36 hours post transfection. Mid-plane images were taken by laser scanning confocal microscopy. Cells grown in DMEM containing 25 mM glucose showed enhanced intracellular fluorescence indicating intracellular retention (FIG. 1A). Similarly, mouse primary beta cells transfected with GLP-1R GFP and GIPR-GFP showed enhanced intracellular fluorescence in high glucose RPMI medium (12 mM glucose) (FIG. 1B). Mean fluorescence ratio at the membrane to cytosol from cells expressing GLP-1RGFP low glucose shows 2-2.2 fold increase compared to cells exposed to high glucose, indicating enhanced membrane fluorescence in low glucose concentrations (FIG. 1C). Mean fluorescence at the membrane and the cytosol from 10 MIN6 cells and 3 primary cells were calculated by drawing ROI of 0.3-0.4 μm diameter. Error bars-mean±SD, Student's t-test ***P<0.0007.

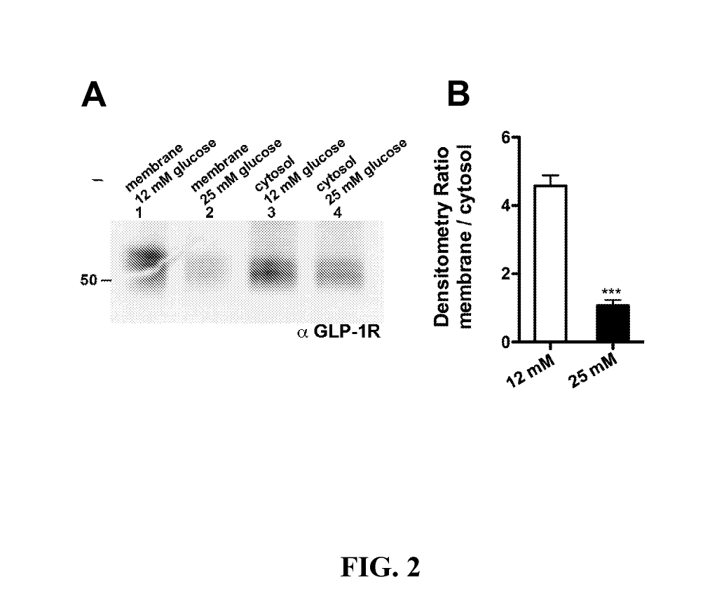

Native plasma membrane bound GLP-1R was quantified in a western blot after a biotinylation assay designed to detect the receptor density at the cell surface. MIN6 cells grown in 12 and 25 mM glucose were biotinylated, lysed and biotinylated membrane proteins were purified with streptavidin beads. Biotinylated receptors at the membrane and non-biotinylated receptors in the cytosol were detected by an anti-GLP-1R antibody. Lanes 1 & 2-GLP-1R in the membrane 12 and 25 mM glucose respectively, Lane 3, 4 Cell lysate from conditions 1 & 2 (FIG. 2A). Ratio GLP-1R protein in the membrane to the cytosol from the same cells. MIN6 cells grown in 25 mM glucose shows an approximately 4.5-fold reduction in plasma membrane bound GLP-1R compared to cells in 12 mM glucose (FIG. 2B). Error bars-mean±SD Student's t-test, **P<0.0014, n=3. Densitometry by ImageJ software.

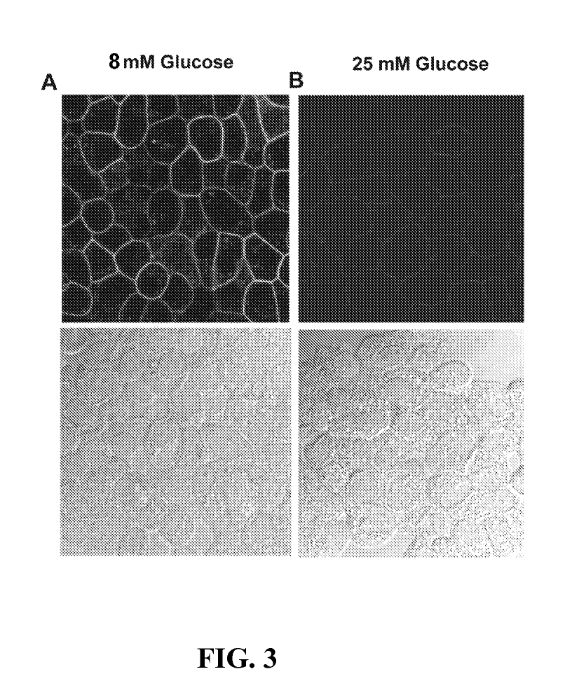

MIN6 insulinoma cells were fixed with 2% paraformaldehyde and treated with fluorescein tagged Exendin 4. The cell surface receptors that bind to the fluorescent agonist (Exendin 4, green) can be detected by this method. Scale bar: 5 μm. Fluorescence imaging of treated cells evidences a decrease in cell surface GLP-1R in 25 mM glucose compared to control 8 mM glucose condition (FIG. 3A-B).

### Example 2

### Incretin Receptor Trafficking to the Cell Membrane of Macrophages and Human Monocytic THP-1 Cells is Glucose Dependent

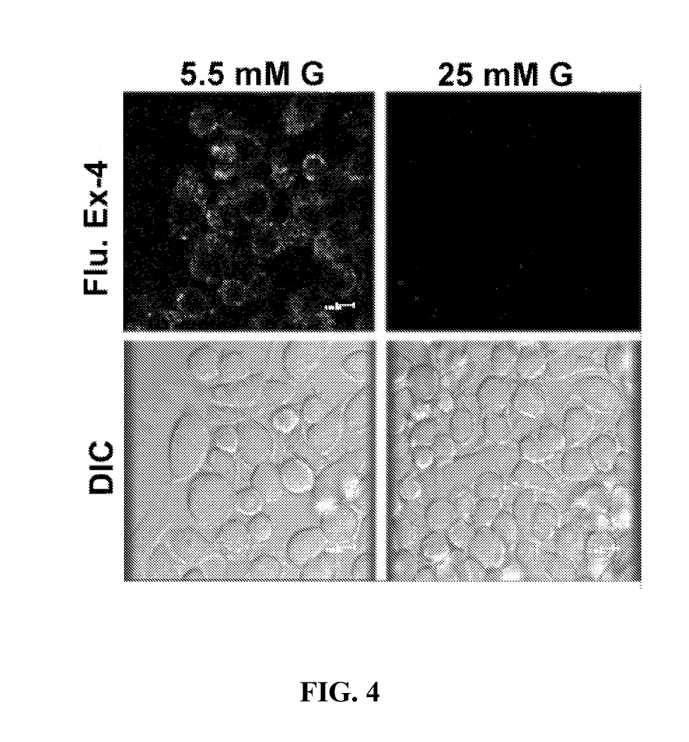

Macrophages cells (RAW) cultured in either normal glucose (5.5 mM) or high glucose (25 mM) over-night and then incubated with 100 nM fluorescein-Trp25-exendin-4 for 15 min, at 21° C. Cells were fixed and imaged by confocal microscopy. Representative, identically exposed confocal mid-plane images are shown. Fluorescence imaging of treated cells evidences a decrease in cell surface GLP-1R in 25 mM glucose compared to control 5.5 mM glucose condition (FIG. 4).

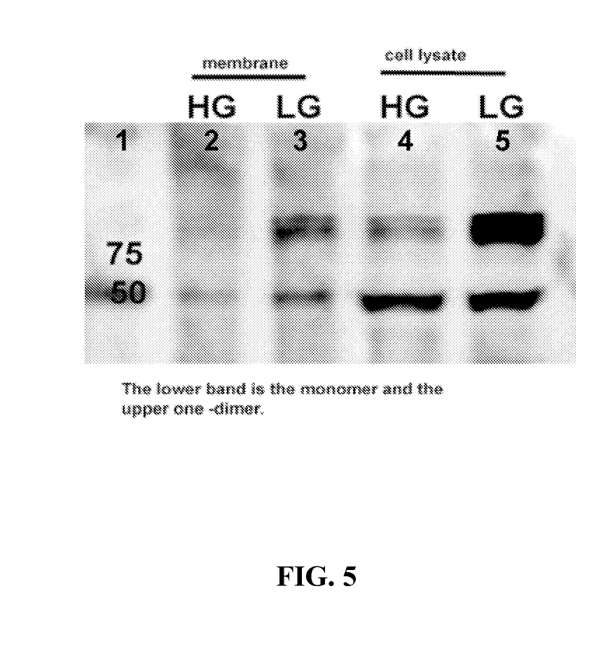

In macrophage cells grown in high glucose conditions cell surface biotinylation assays show that endogenous GLP-1R density at the cell membrane is reduced (FIG. 5). Lane 1-molecular weight marker, Lane 2: cell membrane high glucose (HG-25 mM), Lane 3: cell membrane low glucose (6 mM LG), Lane 4: Cell lysate from Lane 2, Lane 5: Cell lysate from Lane 3. A representative western blot is shown.

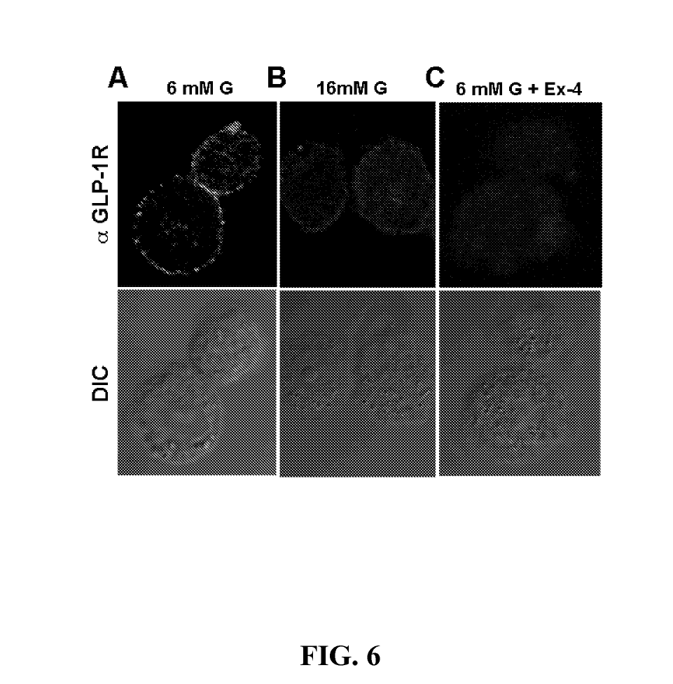

Human monocytic THP-1 cells were cultured in low glucose (6 mM) (FIG. 6A), high glucose (16 mM) (FIG. 6B) or in the presence of Exendin-4, a GLP-1R agonist, in low glucose (FIG. 6C) for 24 hours. Non-permeabilized cells were stained with an anti-GLP-1R antibody raised against the extracellular N-terminus. Representative, identically exposed confocal mid-plane images are shown.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### Example 3

### Rat Peripheral Blood Monocytes Show Glucose Dependent Cell Surface Expression of GLP-1 Receptor

Methods: Peripheral blood cells from Wistar rats were isolated by density separation with Histopaq 1077 (Sigma-Aldrich, St. Louis, Mo.) and monocytes were purified by a Monocyte isolation kit (Stem cell technologies Inc. Vancouver, Canada). Monocytes were maintained in 6 mM or 16 for 12-14 hours. GLP-1 receptor protein from monocyte cell lysate was detected by anti-GLP-1R antibody (Abcam, Cambridge, Mass.). B. A fluorescent GLP-1R agonist, Exendin-4 binding assay was developed to quantify the cell surface receptor density. Cells were fixed with 2% paraformaldehyde for 5 minutes and treated with 50 nM of fluorescein tagged Exendin-4 (Ana Spec, Fremont, Calif.) for 30 minutes. Bound agonist was detected by horse radish peroxidase conjugated anti-fluorescein antibody (Jackson ImmunoResearch, West Grove, Pa.) and a colorimetric peroxidase substrate (Roche, Branchburg, N.J.). The receptor density was quantified by normalizing the absorbance values by DAPI DNA binding dye (Invitrogen, Carlsbad, Calif.) fluorescence.

Cell surface expression of GLP-1 receptor in primary monocytic cells is glucose dependent similar to THP-1 monocytic cells. GLP-1 receptor protein was detected in monocyte cell lysate. A novel colorimetric agonist binding assay was developed to quantify cell surface density of native GLP-1R using a fluorescein tagged Exendin-4 that was reported to bind to endogenous GLP-1R (Chicchi et al., 1997). Fluorescein tagged Exendin-4 was able to bind to paraformaldehyde fixed cells. High glucose concentrations of 16 mM that mimic diabetic conditions resulted in 2.3 fold decrease in endogenous GLP-1R density at the cell membrane compared to the cells cultured in 6 mM glucose. This result indicates that the cell surface GLP-1 receptor density in primary monocytes is glucose dependent as seen is pancreatic beta cells.

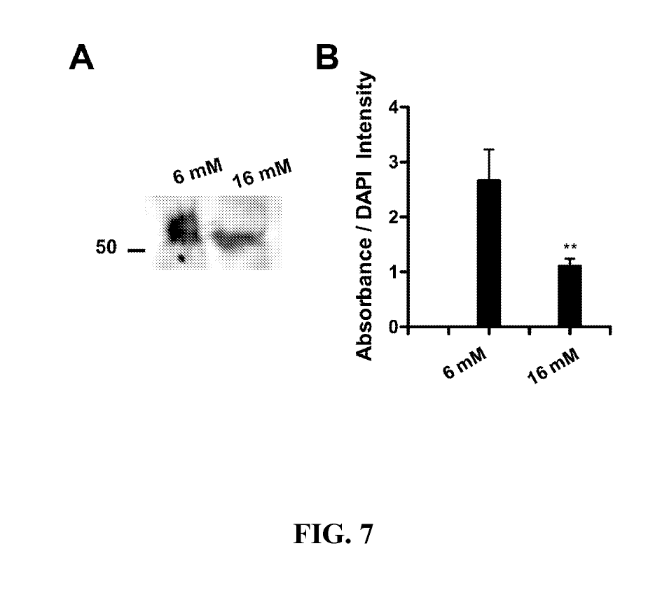

Rat peripheral blood monocytes show glucose dependent cell surface expression of GLP-1 receptor. Western blot shows GLP-1 receptor protein detected by anti-GLP-1 receptor antibody in the whole cell lysate from rat monocytes (FIG. 7A). A colorimetric fluorescein tagged Exendin-4 binding assay shows a 2.3 fold decrease in cell surface GLP-1 receptor density when maintained in 16 mM glucose for 12-14 hours compared to the cells in 6 mM glucose (FIG. 7B). Bound agonist was detected by horse radish peroxidase conjugated anti-fluorescein antibody and a colorimetric substrate. The graph shows absorbance at 450 normalized to the DAPI fluorescence.

### REFERENCES

The following references, to the extent that they provide exemplary procedural or other details supplementary to those set forth herein, are specifically incorporated herein by reference.
Aso, et al., Trans' Res. 159:25-31, 2012.
Chicchi, et al. Peptides. 18:319-321, 1997.
Deiss, et al., Dtsch Med Wochenschr. 136:1116-1120, 2011.
Drucker, et al., Cell Metab. 3:153-165, 2006.
Drucker, et al., J Biol Chem. 262:15659-15665, 1987.
Drucker, et al., Lancet. 368:1696-1705, 2006.
Fritsche, et al., Diabetologia. 43:852-858, 2000.
Holst, et al., Diabetologia. 48:612-615, 2005.
Murphy, et al., J Immunol Methods. 162:211-223, 1993.
Nauck, et al., Acta Diabetol. 30:39-45, 1993.
Nauck, et al., Am J Med. 124:S3-18, 2011.
Rajan, et al., Am J Physiol Endocrinol Metab. 302(6):E714-23, 2012.
Salvi, et al., Cancer Res. 55(15):3374-3379, 2012.
Shiraishi, et al., Biochem Biophys Res Commun. 425:304-308, 2012.
Varanasi, et al., Eur J Endocrinol. 165:77-84, 2011.
Xu, et al., Diabetes. 56:1551-1558, 2007.

1. A method of treating a diabetic patient with an incretin-based therapy comprising:
a) evaluating a measured expression level of GLP-1R in the patient; and,
b) administering the incretin-based therapy using the evaluation to determine the therapy.

2. The method of claim 1, wherein the biological sample is a blood sample.

3. The method of claim 1, wherein the patient has not been previously evaluated for the measured expression level of GLP-1R.

4. The method of claim 3, wherein evaluating the measured expression level of GLP-1R comprises comparing the measured expression level to a representative expression level of GLP-1R that is representative of patients responsive to incretin-based therapy.

5.-6. (canceled)

7. The method of claim 1, wherein evaluating the measured expression level of GLP-1R in the patient comprises comparing the expression level of GLP-1R in the patient to a previously measured expression level of GLP-1R in the patient.

8.-10. (canceled)

11. The method of claim 7, wherein the incretin-based therapy that is administered to the patient is maintained relative to the incretin-based therapy that was administered before the measured expression level was evaluated.

12. The method of claim 1, wherein the incretin-based therapy is an incretin agonist.

13. The method of claim 12, wherein the incretin agonist is a GLP-1 mimetic.

14. The method of claim 2, wherein the blood sample is enriched for peripheral blood monocytes before measuring the expression level of GLP-1R.

15. The method of claim 1, wherein the diabetic patient has Type 1 diabetes.

16. The method of claim 1, wherein the diabetic patient has Type 2 diabetes.

17. A method for treating a patient diagnosed with diabetes or with symptoms of diabetes comprising administering to the patient an incretin-based therapy, wherein:
a) GLP-1R expression has been measured in a biological sample from the patient to obtain a GLP-1R reference level;
b) the GLP-1R reference level was compared to a GLP-1R control level;
and,
c) the patient was determined to have an expression level of GLP-1R that renders the patient likely responsive to the incretin-based therapy.

18.-36. (canceled)

37. A method for treating a diabetic patient or a patient showing signs of diabetes comprising administering an incretin-based therapy to the patient after
GLP-1R expression in peripheral blood monocyte cells from the patient has been measured, wherein the patient has
an expression level of GLP-1R that renders the patient likely responsive to an incretin-based therapy.

38. The method of claim 37, wherein patient has Type 1 diabetes.

39. The method of claim 37, wherein expression of GLP-1R is GLP-1R protein expression.

40. The method of claim 39, wherein GLP-1R protein expression is peripheral blood monocyte surface expression.

41.-45. (canceled)

46. The method of claim 37, wherein measuring expression of GLP-1R comprises flow cytometry.

47. (canceled)

48. The method of claim 37, wherein measuring expression of GLP-1R comprises an ELISA assay, a western blot assay, or slot blot assay.

49. The method of claim 37, wherein expression of GLP-1R is GLP-1R transcript expression.

50.-54. (canceled)

55. The method of claim 17, wherein the patient has been diagnosed with or has signs of Type 2 diabetes.
